(19) United States
(12) Patent Application Publication
(10) Pub. No.: US 2021/0085158 A1
(43) Pub. Date: Mar. 25, 2021

(54) ENDOSCOPE, ENDOSCOPE SYSTEM, INSERTION PORTION OF ENDOSCOPE, AND CALCULUS COLLECTING METHOD

(71) Applicant: OLYMPUS CORPORATION, Tokyo (JP)

(72) Inventors: Soichi IKUMA, Akishima-shi (JP); Eijiro SATO, Hachioji-shi (JP)

(73) Assignee: OLYMPUS CORPORATION, Tokyo (JP)

(21) Appl. No.: 17/021,567

(22) Filed: Sep. 15, 2020

Related U.S. Application Data
(63) Continuation of application No. PCT/JP2018/042193, filed on Nov. 14, 2018.
(60) Provisional application No. 62/643,815, filed on Mar. 16, 2018.

(51) Int. Cl. A61B 1/00; A61B 1/015; A61B 1/05

(52) U.S. Cl. CPC A61B 1/00119; A61B 1/015; A61B 1/00068; A61B 1/05; A61B 1/00094

(57) ABSTRACT

An endoscope includes an insertion portion including a first conduit for delivering a fluid, and a second conduit for suctioning a fluid. A distal end opening of the second conduit may have an opening area larger than an opening area of a distal end opening of the first conduit, and in the second conduit, an inside diameter on a distal end side may be smaller than an inside diameter on a proximal end side. In a calculus collecting method, fluid can be delivered into the subject from the first conduit, and fluid can be suctioned into the second conduit. Intermittently, fluid delivery from the first conduit may be stopped so that fluid can be delivered through the second conduit.

### CROSS REFERENCE TO RELATED APPLICATION

This application is a continuation application of PCT/JP2018/042193 filed on November 14, 2018 and claims benefit of U.S. Provisional Patent Application No. 62/643,815 filed in the U.S.A. on Mar. 16, 2018, the entire contents of which are incorporated herein by this reference.

### BACKGROUND

### 1. Technical Field

The present disclosure relates to an endoscope, an endoscope system, an insertion portion of the endoscope, and a calculus collecting method that are capable of collecting calculus fragments or the like in a subject.

### 2. Description of the Related Art

Various calculi, polyps and the like that are found in examinations or the like are collected. For example, in the case of a renal calculus, by a ureteroscopic procedure (ureteroscopy), a flexible endoscope is inserted into a kidney pelvis from a ureter, and the calculus is crushed by using a laser treatment instrument inserted through a treatment instrument insertion channel.

The crushed calculus fragments crushed by the laser is expected to be left in a kidney calyx and discharged naturally to outside of the subject, and is left as it is, and when the sizes of the crushed calculus fragments are large, the crushed calculus fragments are collected by basket forceps.

As an apparatus that performs collection of living tissue and the like of polyps, there is a debrider or a morcellator that sucks lesion parts while morcellating and removing the lesion parts.

As an apparatus that simply collects crushed calculus fragments without using basket forceps, there is also proposed an apparatus that collects a calculus by injecting a fluid into a kidney pelvis.

### SUMMARY

An endoscope according to one aspect of the present disclosure includes an insertion portion configured to be inserted into a subject, a first conduit that includes a first distal end opening provided in a distal end portion of the insertion portion, is disposed along a longitudinal axis of the insertion portion from the distal end portion of the insertion portion, and is capable of delivery of a fluid, and a second conduit that includes a second distal end opening provided in the distal end portion of the insertion portion and different from the first distal end opening, is disposed along the longitudinal axis of the insertion portion from the distal end portion of the insertion portion, and is capable of suction of the fluid, wherein the second distal end opening has an opening area larger than an opening area of the first distal end opening, and in the second conduit, an inside diameter on a distal end side is smaller than an inside diameter on a proximal end side.

An endoscope system of one aspect of the present disclosure includes the endoscope of the one aspect of the present disclosure, and a processor configured to control a first pump configured to perform delivery of the fluid to the second conduit and a second pump configured to perform suction of the fluid from the third conduit.

An insertion portion of an endoscope of one aspect of the present disclosure is an insertion portion configured to be inserted into a subject, and includes a first conduit that includes a first distal end opening provided in a distal end portion of the insertion portion, is disposed along a longitudinal axis of the insertion portion from the distal end portion of the insertion portion, and is capable of delivery of a fluid, and a second conduit that includes a second distal end opening provided in the distal end portion of the insertion portion and different from the first distal end opening, is disposed along the longitudinal axis of the insertion portion from the distal end portion of the insertion portion, and is capable of suction of the fluid, wherein the second distal end opening has an opening area larger than an opening area of the first distal end opening, and in the second conduit, an inside diameter on a distal end side is smaller than an inside diameter on a proximal end side.

A calculus collecting method of one aspect of the present disclosure is a method for collecting a calculus in a subject by using a first conduit configured to be inserted into the subject to perform delivery of a fluid, and a second conduit configured to be inserted into the subject to perform suction of the fluid and the calculus, wherein a first operation of delivering a fluid into the subject from the first conduit, and performing suction of a fluid from the second conduit, and a second operation of stopping delivery of the fluid from the first conduit, and performing delivery of the fluid from the second conduit are repeated so that the first operation becomes longer than the second operation.

### BRIEF DESCRIPTION OF THE DRAWINGS

FIG. 1 is a configuration diagram of an endoscope system relating to an embodiment;

FIG. 2 is a configuration diagram of a distal end portion of an insertion portion as the distal end portion of the insertion portion is seen in a distal end direction of a longitudinal axis of the insertion portion, relating to the embodiment;

FIG. 3 is a configuration diagram of the distal end portion of the insertion portion formed by using a multi-lumen tube, as the distal end portion of the insertion portion is seen in the distal end direction of the longitudinal axis of the insertion portion, relating to the embodiment;

FIG. 4 is a schematic configuration diagram illustrating a configuration of the insertion portion relating to the embodiment;

FIG. 5 is a flowchart illustrating an example of a flow of processing for operations of water feeding and suction in a control unit, relating to the embodiment;

FIG. 6 is a view for explaining a flow of a physiological saline solution discharged from a distal end opening of a water feeding tube, relating to the embodiment;

FIG. 7 is a schematic configuration diagram illustrating a configuration of a distal end portion of an insertion portion relating to modification 1 of the embodiment;

FIG. 8 is a schematic configuration diagram illustrating a configuration of an insertion portion relating to modification 2 of the embodiment;

FIG. 9 is a sectional view of a distal end portion of a water feeding tube along a longitudinal axis direction of the water feeding tube, relating to modification 2 of the embodiment;

FIG. 10 is a side view of a distal end portion of a water feeding tube seen in a direction orthogonal to a longitudinal axis of the water feeding tube relating to modification 3 of the embodiment;

FIG. 11 is a front view of the distal end portion of the water feeding tube seen in a longitudinal axis direction of the water feeding tube relating to modification 3 of the embodiment;

FIG. 12 is a sectional view of a connection portion of a distal end rotation portion and the water feeding tube along the longitudinal axis direction of the water feeding tube relating to modification 3 of the embodiment;

FIG. 13 is a schematic configuration diagram illustrating a configuration of an insertion portion in which an inside diameter of a distal end portion of a water feeding tube is made smaller than an inside diameter of a proximal end portion, relating to modification 4 of the embodiment;

FIG. 14 is a schematic configuration diagram illustrating a configuration of an insertion portion relating to modification 6 of the embodiment;

FIG. 15 is a schematic configuration diagram illustrating a configuration of an insertion portion relating to modification 7 of the embodiment;

FIG. 16 is a front view illustrating a shape of a distal end opening of a suction tube, relating to modification 8 of the embodiment;

FIG. 17 is a front view of a distal end opening of a suction tube, relating to modification 9 of the embodiment;

FIG. 18 is a front view of the distal end opening of the suction tube, relating to modification 9 of the embodiment;

FIG. 19 is a view illustrating a propeller provided in a suction tube, relating to modification 11 of the embodiment;

FIG. 20 is a time chart of operations of a stop valve and a three-way stopcock for intermittent suction of suction, relating to modification 12 of the embodiment;

FIG. 21 is a time chart of operations of feeding and suction, relating to modification 13 of the embodiment;

FIG. 22 is a configuration diagram of a distal end portion of an endoscope insertion portion as the distal end portion of the endoscope insertion portion is seen in a distal end direction of a longitudinal axis of the endoscope insertion portion as an insertion portion, relating to modification 14 of the embodiment;

FIG. 23 is a configuration diagram of an endoscope system relating to modification 18; and

FIG. 24 is a configuration diagram of a distal end portion of an insertion portion, as the distal end portion of the insertion portion is seen in a distal end direction of a longitudinal axis of the insertion portion, relating to modification 18.

### DETAILED DESCRIPTION OF THE PREFERRED EMBODIMENTS

Hereinafter, the present endoscope, insertion portion thereof, endoscope system, and related methods will be described with use of an embodiment.

(System configuration)

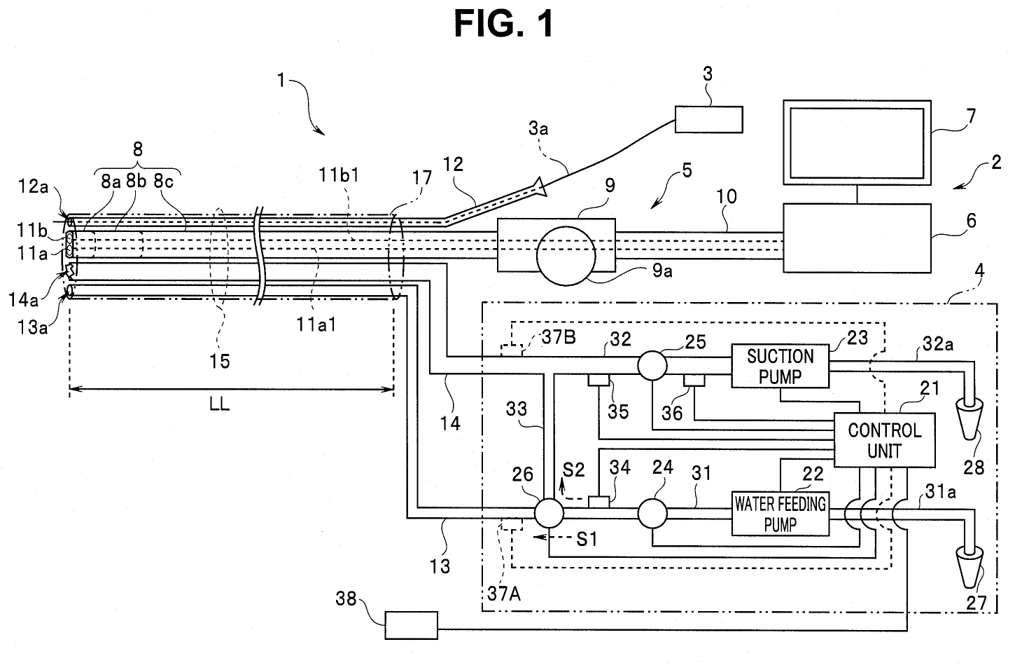

FIG. 1 is a configuration diagram of an endoscope system relating to the embodiment of the present disclosure.

The endoscope system 1 is configured by having an endoscope apparatus 2, a laser apparatus 3, and a water feeding and suction apparatus 4. The endoscope apparatus 2 has an endoscope 5, a main body apparatus 6, and a display apparatus 7 connected to the main body apparatus 6. A laser probe 3a is extended from the laser apparatus 3.

The endoscope system 1 is used to observe an inside of a subject, and to crush and collect a calculus or the like inside a subject, as described later.

The endoscope apparatus 2 displays an image of an inside of a subject on the display apparatus 7, and a surgeon can observe the inside of the subject, and perform a necessary treatment, while looking at the endoscope image displayed on the display apparatus 7.

Here, the laser apparatus 3 generates a laser light for crushing a calculus or the like. The generated laser light passes through an inside of the laser probe 3a, is emitted from a distal end of the laser probe 3a, and crushes the calculus or the like. The surgeon can apply the laser light to the calculus or the like while looking at the endoscope image displayed on the display apparatus 7.

The water feeding and suction apparatus 4 feeds a physiological saline solution into a subject, and sucks the physiological saline solution in the subject.

For this purpose, the water feeding and suction apparatus 4 has a control unit 21, a water feeding pump 22, and a suction pump 23. A physiological saline solution discharged from the water feeding pump 22 is supplied into a subject via a water feeding tube 13 described later. The physiological saline solution in the subject is sucked by the suction pump 23 via a suction tube 14.

By the water feeding and suction apparatus 4, a fresh physiological saline solution is always supplied into the subject, an inside of the subject is filled with a physiological saline solution, and the physiological saline solution in the subject is sucked.

Accordingly, in a state where a physiological saline solution such as a physiological saline solution is sucked from the inside of the subject while a physiological saline solution is supplied into the subject, a surgeon can perform crush of a calculus by the laser light and collection of the calculus while looking at an endoscope image of the inside of the subject.

(Configuration of endoscope apparatus)

The endoscope 5 of the endoscope apparatus 2 has an elongated endoscope insertion portion 8, an operation portion 9, and a universal cable 10 extended from the operation portion 9.

The endoscope insertion portion 8 has a distal end portion 8a, a bending portion 8b, and a flexible tube portion 8c, from a distal end. The distal end portion 8a has an observation window 11a, and two illumination windows 11b on a distal end surface.

At a rear side of the observation window 11a, a distal end surface of an elongated image guide 11a1 of an optical fiber bundle is placed. Therefore, at a distal end of the image guide 11a1, an objective optical system such as a lens is placed, and a distal end portion of the objective optical system configures the observation window 11a.

The image guide 11a1 is inserted through insides of the endoscope insertion portion 8, the operation portion 9, and the universal cable 10. A proximal end portion of the image guide 11a1 is connected to a connector provided at an end portion of the universal cable 10. When the connector of the universal cable 10 is connected to the main body apparatus 6, a light emitted from a proximal end surface of the image guide 11a1 is irradiated to a light receiving surface of an image pickup device in the main body apparatus 6.

Note that here, the image guide 11a1 is provided behind the observation window 11a, but an image pickup device such as a CMOS image sensor may be provided. In that case, a signal line extended from the image pickup device is inserted through the insides of the endoscope insertion portion 8, the operation portion 9, and the universal cable 10, and an image pickup signal of the image pickup device is supplied to an image processing circuit of the main body apparatus 6.

A distal end surface of a light guide 11b1 of an optical fiber bundle is placed at a rear side of the illumination window 11b. Therefore, an illumination optical system such as a lens is placed at a distal end of the light guide 11b1, and a distal end portion of the illumination optical system configures the illumination window 11b.

The elongated light guide 11b1 configures an illumination unit. A proximal end portion of the light guide 11b1 is connected to the aforementioned connector provided at the end portion of the universal cable 10, and guides an illumination light from a light source in the main body apparatus 6. The illumination light is emitted from the illumination window 11b.

Note that here, behind the illumination window 11b, the light guide 11b1 is provided, but a light emitting element such as a light emitting diode (LED) may be provided. In that case, a power supply line extending from the light emitting element is inserted through the insides of the endoscope insertion portion 8, the operation portion 9, and the universal cable 10.

The bending portion 8b includes a plurality of bending pieces, and is bendable in a predetermined direction, for example, an up-down direction. A distal end portion of a bending wire not illustrated is fixed to a distal end bending piece, and a proximal end portion of the bending wire is connected to a bending knob 9a of the operation portion 9. The surgeon operates the bending knob 9a, and thereby the bending portion 8b bends.

The flexible tube portion 8c is configured by a flex, a braid, and a skin resin being stacked in layer, from an inner side. The flex is a spiral tube as a flexible member having a shape in which a flat plate material is spirally wound. The braid is a metallic net tube. The skin resin is formed on an outer peripheral portion of the braid so that a part of the skin resin is inserted between metal element wires of the braid. Therefore, the flexible tube portion 8c has some degree of rigidity and flexibility.

Note that though not illustrated, the operation portion 9 is provided with various operation buttons such as a release button that instructs to record an endoscope image.

The main body apparatus 6 is a video processor having a driving circuit configured to drive the image pickup device, and an image processing circuit configured to receive an image pickup signal from the image pickup device and generate an image signal.

The main body apparatus 6 also contains a light source for an illumination light. A light from the light source is incident on a proximal end surface of the aforementioned light guide 11b1, passes through the light guide 11b1, and is emitted from the illumination window 11b.

The display apparatus 7 receives the image signal from the main body apparatus 6, and displays an endoscope image on a screen. Therefore, a surgeon or the like can perform an examination and a treatment by looking at the endoscope image displayed on the display apparatus 7.

(Configuration of laser treatment apparatus)

The laser apparatus 3 generates a laser light that crushes a calculus here. The laser probe 3a is extended from the laser apparatus 3. The laser probe 3a and the laser apparatus 3 configure a laser treatment apparatus as a surgical treatment apparatus.

The laser probe 3a is configured to be insertable into a channel tube 12.

The channel tube 12 is a conduit member having a conduit through which the laser probe 3a can be inserted. The channel tube 12 is placed on an outer peripheral portion of the suction tube 14 described later so that a longitudinal axis of the channel tube 12 and a longitudinal axis of the endoscope insertion portion 8 are parallel with each other.

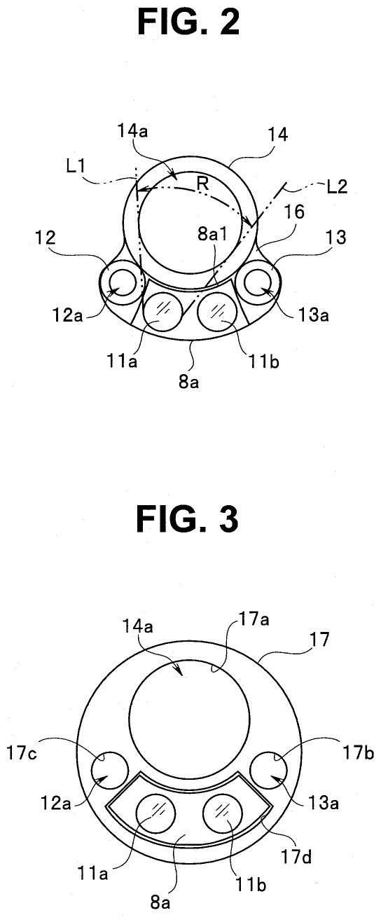

The surgeon inserts the laser probe 3a into the channel tube 12, and can crush a calculus or the like by emitting a laser light in a state where a distal end portion of the laser probe 3a is protruded from a distal end opening 12a (FIG. 2) of the channel tube 12.

Note that here, the laser apparatus 3 is used to crush a calculus or the like, but an electric knife apparatus or the like can be used in a case of excision of tumors or the like.

(Configuration of insertion portion)

The water feeding tube 13 and the suction tube 14 that are connected to the water feeding and suction apparatus 4 are placed in parallel with the longitudinal axis of the endoscope insertion portion 8 similarly to the channel tube 12. In other words, the water feeding tube 13 and the suction tube 14 are placed so that a longitudinal axis of the water feeding tube 13 and a longitudinal axis of the suction tube 14 are parallel with the longitudinal axis of the endoscope insertion portion 8.

The endoscope insertion portion 8, the channel tube 12, the water feeding tube 13, and the suction tube 14, which are in a bundled state, are closely attached and fixed to one another by fixing means such as an adhesive to form one insertion portion 15. The fixing means such as an adhesive is provided in a range LL corresponding to a length inserted into a subject, in the insertion portion 15. Therefore, in the range LL, four longitudinal axes of the endoscope insertion portion 8, the channel tube 12, the water feeding tube 13, and the suction tube 14 are parallel with one another.

Therefore, parts corresponding to the range LL of the endoscope insertion portion 8, the channel tube 12, the water feeding tube 13, and the suction tube 14 that are bundled configure the insertion portion 15 that is inserted into a subject.

A distal end portion of the insertion portion 15 has the observation window 11a for observing a subject, and the illumination windows 11b for illuminating the subject.

Note that here, the fixing means is an adhesive, but may be heat-shrinkable tubing or the like. For example, the endoscope insertion portion 8, the channel tube 12, the water feeding tube 13, and the suction tube 14 are inserted into a heat-shrinkable tubing, and thereafter are closely attached and fixed to one another by the heat-shrinkable tubing being heated from an outer peripheral portion.

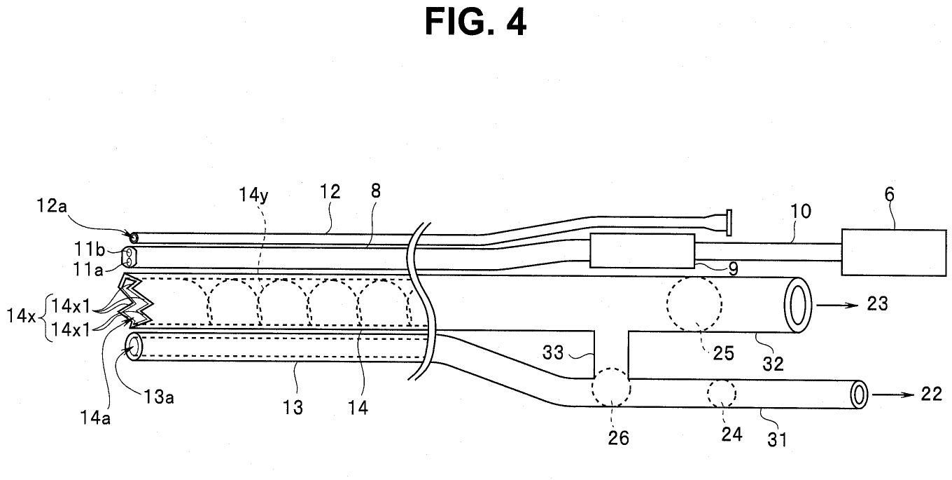

A spiral groove 14y in a longitudinal axis direction of the suction tube 14 is formed on an inner peripheral surface of the suction tube 14 (FIG. 4). A sucked physiological saline solution flows in a vortex shape inside the suction tube 14 by the groove 14y. In other words, the suction tube 14 is formed to generate a vortex flow inside.

The vortex flow has a low pressure in a vicinity of a center of the vortex, that is, in a vicinity of a center axis of the suction tube 14, and has a high pressure in a vicinity of an outside of the vortex, that is, in a vicinity of an inner wall of the suction tube 14. As a result, crushed calculus fragments easily flow in the vicinity of the center axis of the suction tube 14, and therefore, the crushed calculus fragments are less likely to get stuck in the suction tube 14.

FIG. 2 is a configuration diagram of the distal end portion of the insertion portion 15 as the distal end portion of the insertion portion 15 is seen in a distal end direction of a longitudinal axis of the insertion portion 15.

The distal end portion 8a of the endoscope insertion portion 8 has a bent rectangular shape when seen in the distal end direction of the longitudinal axis of the insertion portion 15. In other words, the endoscope insertion portion 8 has a partial cylindrical shape that is curved along the outer peripheral surface of the suction tube 14. A distal end opening 14a of the suction tube 14 is disposed to contact a surface 8a1 on a bent side of the distal end portion 8a.

The distal end opening 12a of the channel tube 12 is disposed to contact the outer peripheral surface of the suction tube 14 and one end of the distal end portion 8a.

A distal end opening 13a of the water feeding tube 13 is disposed to contact the outer peripheral surface of the suction tube 14 and the other end of the distal end portion 8a.

An opening diameter of the distal end opening 14a of the suction tube 14 is larger than an opening diameter of the distal end opening 13a of the water feeding tube 13.

As described later, the water feeding pump 22 and the suction pump 23 are controlled so that a water feeding amount of the water feeding tube 13 and a suction amount of the suction tube 14 are equal to each other. Accordingly, the opening diameter of the distal end opening 14a of the suction tube 14 is larger than the opening diameter of the distal end opening 13a of the water feeding tube 13, and therefore, a moving speed of the physiological saline solution in the suction tube 14 is lower than a moving speed of the physiological saline solution in the water feeding tube 13.

The endoscope insertion portion 8, the channel tube 12, the water feeding tube 13, and the suction tube 14 are closely attached and fixed to one another by an adhesive 16 so that the distal end portion 8a of the endoscope insertion portion 8, the distal end opening 12a of the channel tube 12, and the distal end opening 13a of the water feeding tube 13 are disposed on an outer peripheral portion of the distal end opening 14a of the suction tube 14, as illustrated in FIG. 2.

As above, the channel tube 12 has the distal end opening 12a, is disposed along the longitudinal axis of the insertion portion 15 from the distal end portion of the insertion portion 15, and configures a conduit through which a long member such as the laser probe 3a is insertable.

The water feeding tube 13 has the distal end opening 13a different from the distal end opening 12a, in the distal end portion of the insertion portion 15, the distal end opening 13a has a first opening area, the water feeding tube 13 is disposed along the longitudinal axis of the insertion portion 15 from the distal end portion of the insertion portion 15, and configures a conduit capable of delivery of a fluid.

The suction tube 14 has the distal end opening 14a different from the distal end opening 12a and the distal end opening 13a, the distal end opening 14a has a second opening area larger than the first opening area, and the suction tube 14 is disposed along the longitudinal axis of the insertion portion 15 from the distal end portion of the insertion portion 15, and configures a conduit capable of suction of a fluid.

By doing so, an entire insertion diameter can be reduced to be small while the large second opening area required to collect large crushed calculus fragments is realized. By adopting the small first opening area, it is possible to obtain a high feeding speed even with a small water feeding amount and cause crushed calculus fragments to fly up, so that collection efficiency is improved. Even when a same suction amount as the water feeding amount is realized to keep a pressure inside the subject constant, it is also possible to reduce a suction force since the second opening area is large, and it is possible to avoid or reduce a damage when a mucous membrane or the like on a subject surface is sucked. It is possible to realize a high feeding speed and a low suction force while saving a total amount of the physiological saline solution that is used.

The distal end opening 12a is disposed in the distal end portion of the insertion portion 15 so that the distal end opening 12a is not located in a region within a range R sandwiched by two virtual tangent lines L1 and L2 that are from an outer periphery of the observation window 11a to the distal end opening 14a of the suction tube 14, when the distal end portion of the insertion portion 15 is seen in a distal end direction of the longitudinal axis of the insertion portion 15, as illustrated in FIG. 2.

By doing so, in an endoscope field of view, a direction of a field of view of the laser probe 3a and an object located at a distal end of the laser probe 3a, and a flow range of crushed calculus fragments that are sucked hardly overlap each other, and an operation of the laser probe 3a is not inhibited. The crushed calculus fragments that are sucked can be restrained from contacting the laser probe 3a, and a damage to the laser probe 3a can be prevented.

Note that the insertion portion 15 may be formed by using a multi-lumen tube.

FIG. 3 is a configuration diagram of the distal end portion of the insertion portion 15 formed by using a multi-lumen tube as the distal end portion of the insertion portion 15 is seen in the distal end direction of the longitudinal axis of the insertion portion 15. In FIG. 1, the multi-lumen tube 17 is shown by an alternate long and short dashes line. The multi-lumen tube 17 is made of a resin, and has a length of at least the aforementioned range LL.

One hole 17a of the multi-lumen tube 17 configures an inner space of the suction tube 14, and a distal end opening of the hole 17a corresponds to the distal end opening 14a of the suction tube 14.

Another hole 17b of the multi-lumen tube 17 configures an internal space of the water feeding tube 13, and a distal end opening of the hole 17b corresponds to the distal end opening 13a of the water feeding tube 13.

Still another hole 17c of the multi-lumen tube 17 configures an internal space of the channel tube 12, and a distal end opening of the hole 17c corresponds to the distal end opening 12a of the channel tube 12.

Still another hole 17d of the multi-lumen tube 17 configures an internal space through which the endoscope insertion portion 8 is inserted. The observation window 11a and the illumination window 11b in the distal end portion 8a are placed in a distal end opening of the hole 17d.

(Configuration of water feeding and suction apparatus)

As illustrated in FIG. 1, the water feeding and suction apparatus 4 has the control unit 21, the water feeding pump 22, the suction pump 23, two stop valves 24 and 25, a three-way stopcock 26, a water feeding tank 27, and a suction tank 28.

The control unit 21 includes a processor having a central processing unit (hereinafter, referred to as a CPU), and realizes respective functions of the water feeding and suction apparatus 4 by the CPU reading and executing a program stored in a memory such as a ROM.

The control unit 21 is connected to the water feeding pump 22, the suction pump 23, the two stop valves 24 and 25, and the three-way stopcock 26 by respective signal lines. Accordingly, respective operations of the water feeding pump 22 that performs delivery of a physiological saline solution, the suction pump 23 that performs suction of a physiological saline solution, the two stop valves 24 and 25, and the three-way stopcock 26 are controlled by the control unit 21.

The water feeding pump 22 is connected to a conduit 31. The conduit 31 is connected to the water feeding tube 13 by a connector not illustrated, and the water feeding tube 13 is extended from the water feeding and suction apparatus 4. The water feeding pump 22 is also connected to the water feeding tank 27 by a conduit 31a.

Accordingly, the water feeding pump 22 discharges a physiological saline solution in the water feeding tank 27 from the conduit 31a to the conduit 31.

The stop valve 24 and the three-way stopcock 26 are provided midway in the conduit 31.

The suction pump 23 is connected to a conduit 32. The conduit 32 is connected to the suction tube 14 by a connector not illustrated, and the suction tube 14 is extended from the water feeding and suction apparatus 4. The suction pump 23 is also connected to the suction tank 28 by a conduit 32a.

Accordingly, the suction pump 23 discharges a physiological saline solution sucked from the conduit 32 to the suction tank 28 via the conduit 32a.

The stop valve 25 is provided midway in the conduit 32.

As illustrated in FIG. 1, a conduit 33 is provided between the three-way stopcock 26 and the conduit 32. More specifically, the conduit 33 is a connection portion that connects a proximal end portion of the water feeding tube 13 and a proximal end portion of the suction tube 14. The three-way stopcock 26 is provided in the conduit 33 as the connection portion.

The three-way stopcock 26 can take two states. In a first state (hereinafter, also referred to as a pattern A), the physiological saline solution from the conduit 31 flows to the water feeding tube 13, as shown by a dotted line S1. In a second state (hereinafter, also referred to as a pattern B), the physiological saline solution from the conduit 31 flows to the conduit 33, as shown by a dotted line S2.

A flowmeter 34 is provided at the conduit 31. The flowmeter 34 is connected to the control unit 21 by a signal line, detects a flow rate of the physiological saline solution flowing in the conduit 31, and outputs a detection value to the control unit 21.

Likewise, a flowmeter 35 is provided at the conduit 32. The flowmeter 35 is connected to the control unit 21 by a signal line, detects a flow rate of the physiological saline solution flowing in the conduit 32, and outputs a detection value to the control unit 21.

A pressure gauge 36 is provided at the conduit 31. The pressure gauge 36 is connected to the control unit 21 by a signal line, detects a pressure in the conduit 31, and outputs a detection value to the control unit 21.

A foot switch 38 is connected to the water feeding and suction apparatus 4. The foot switch 38 is operated with a foot by a surgeon, and an operation signal of the foot switch 38 is supplied to the control unit 21 of the water feeding and suction apparatus 4. When the control unit 21 receives an operation signal, the control unit 21 drives the water feeding pump 22 and the suction pump 23, and starts feeding to the conduit 31 and suction from the conduit 32, when the operation signal is an instruction signal to start operation.

The control unit 21 calculates the flow rate, that is, a feeding flow rate of the physiological saline solution flowing in the conduit 31, based on a detection signal from the flowmeter 34. Likewise, the control unit 21 calculates a flow rate, that is, a suction flow rate of the physiological saline solution flowing in the conduit 32, based on a detection signal from the flowmeter 35.

The control unit 21 calculates a water feeding amount and a suction amount in a predetermined time period, and controls the water feeding pump 22 and the suction pump 23 so that the water feeding amount and the suction amount become equal to each other.

As a result, the amount of the physiological saline water in the subject, and the pressure in the subject are kept constant. The pressure in the subject is kept constant, and thereby it is possible to prevent worsening of the field of view of the endoscope image.

Note that the control unit 21 controls the water feeding pump 22 or the suction pump 23 based on a detection signal of the pressure gauge 36, so that the pressure inside the subject does not reach a predetermined value or more for safety of the subject. In other words, the control unit 21 controls the water feeding pump 22 or the suction pump 23 so that the pressure in the suction tube 14 does not rise to a predetermined value or more, based on the pressure inside the suction tube 14.

FIG. 4 is a schematic configuration diagram illustrating a configuration of the insertion portion 15.

A distal end surface of the suction tube 14 has a recessed and protruded portion 14x. The recessed and protruded portion 14x has a plurality of V-shaped cutout portions 14x1. In other words, as illustrated in FIG. 4, the distal end surface of the suction tube 14 is not on one plane when the distal end surface of the suction tube 14 is seen in a direction orthogonal to the longitudinal axis of the suction tube 14.

The recessed and protruded portion 14x is formed on the distal end surface of the suction tube 14 so that crushed calculus fragments do not completely close the distal end opening 14a. In other words, the distal end opening 14a of the suction tube 14 has the recessed and protruded portion 14x.

Accordingly, the distal end opening 14a can have at least one protruded portion or at least one recessed portion in the longitudinal axis direction of the insertion portion 15 so that the crushed calculus fragments do not completely close the distal end opening 14a.

The physiological saline solution discharged from the distal end opening 13a of the water feeding tube 13 is released into the subject, and the physiological saline solution in the subject is sucked into the suction tube 14 from the distal end opening 14a of the suction tube 14.

### (Operation)

Hereinafter, an operation of the endoscope system 1 will be described by citing a case where a renal calculus is crushed, and the crushed calculus fragments are collected as an example.

At first, a surgeon inserts a guide wire into a urethra, and causes a distal end portion of the guide wire to reach a desired site in a kidney. The surgeon inserts an access sheath into a subject along the guide wire.

After extracting the guide wire, the surgeon inserts the aforementioned insertion portion 15 into the access sheath.

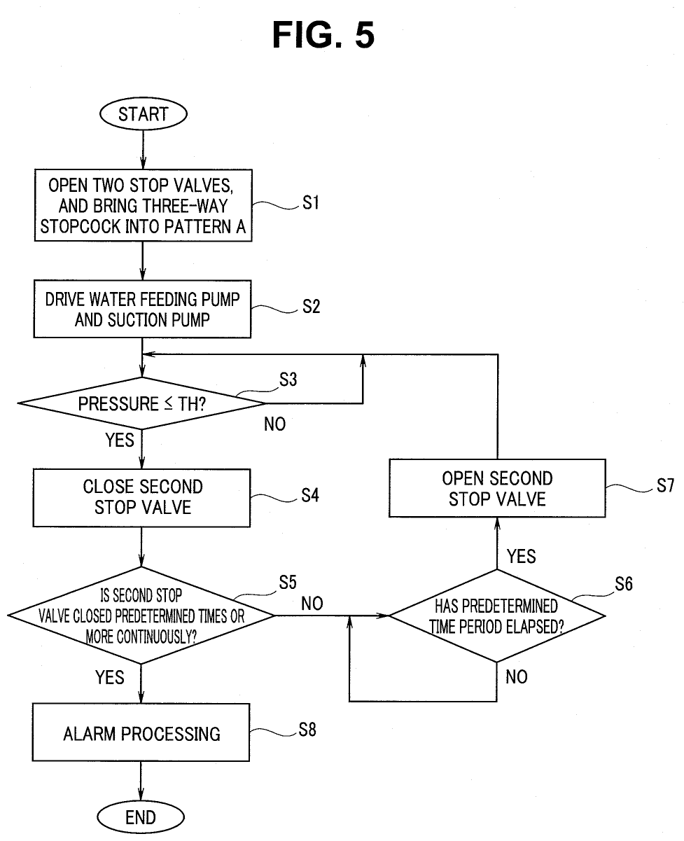

Subsequently, when the surgeon turns on the foot switch 38, processing in FIG. 5 is executed, and the surgeon can collect crushed calculus fragments while crushing the renal calculus. As described later, powdery crushed calculus fragments or very small crushed calculus fragments are collected through the suction tube 14, whereas the crushed calculus fragments sticking to the distal end opening 14a of the suction tube 14 are collected by pulling the insertion portion 15 out of the access sheath.

FIG. 5 is a flowchart illustrating an example of a flow of processing for operations of feeding and suction in the control unit 21.

When the foot switch 38 is turned on, the control unit 21 opens the stop valves 24 and 25, and brings a state of the three-way stopcock 26 into the state of the pattern A (step (hereinafter, abbreviated as S) 1). In other words, the control unit 21 transmits a control signal to open to the stop valves 24 and 25, and transmits a control signal to bring the state of the three-way stopcock 26 into the pattern A to the three-way stopcock 26.

Subsequently, the control unit 21 drives the water feeding pump 22 and the suction pump 23 (S2).

As a result, at a same time as a physiological saline solution such as a physiological saline solution is discharged into the subject from the distal end opening 13a of the water feeding tube 13, the physiological saline solution in the subject is sucked into the distal end opening 14a of the suction tube 14.

When the foot switch 38 is turned on in a state where the distal end portion of the insertion portion 15 is inserted into the kidney, for example, the physiological saline solution is delivered into the kidney, and the physiological saline solution in the kidney is sucked by the suction pump 23.

During an examination, the endoscope apparatus 2 operates, and therefore, an endoscope image is always displayed on the display apparatus 7.

The control unit 21 determines whether or not the pressure in the conduit 32 reaches a predetermined threshold TH or less based on a detection signal of the pressure gauge 36 (S3).

When the pressure in the conduit 32 does not reach the predetermined threshold TH or less (S3: NO), processing in S3 is performed continuously.

For example, when crushed calculus fragments are in a state where the crushed calculus fragments accumulate to close the distal end opening 14a of the suction tube 14, the pressure in the conduit 32 declines, and therefore, it is determined whether or not such a state is brought about based on the pressure in the conduit 32.

While the pressure in the conduit 32 does not reach the predetermined threshold TH or less, the surgeon can insert the laser probe 3a into the channel tube 12, apply the laser light to a calculus, and crush the calculus when the surgeon looks at the endoscope image and finds the calculus during the examination.

When the calculus is crushed by the laser light, the powdery crushed calculus fragments are sucked into the distal end opening 14a of the suction tube 14.

The physiological saline solution is discharged from the distal end opening 13a of the water feeding tube 13. The physiological saline solution in the kidney is sucked into the distal end opening 14a of the suction tube 14, and accumulates in the suction tank 28.

The physiological saline solution discharged from the distal end opening 13a of the water feeding tube 13 becomes a flow.

In particular, the opening diameter of the distal end opening 13a of the water feeding tube 13 is smaller than the opening diameter of the distal end opening 14a of the suction tube 14, and therefore a speed of a flow discharged from the distal end opening 13a of the water feeding tube 13 is high.

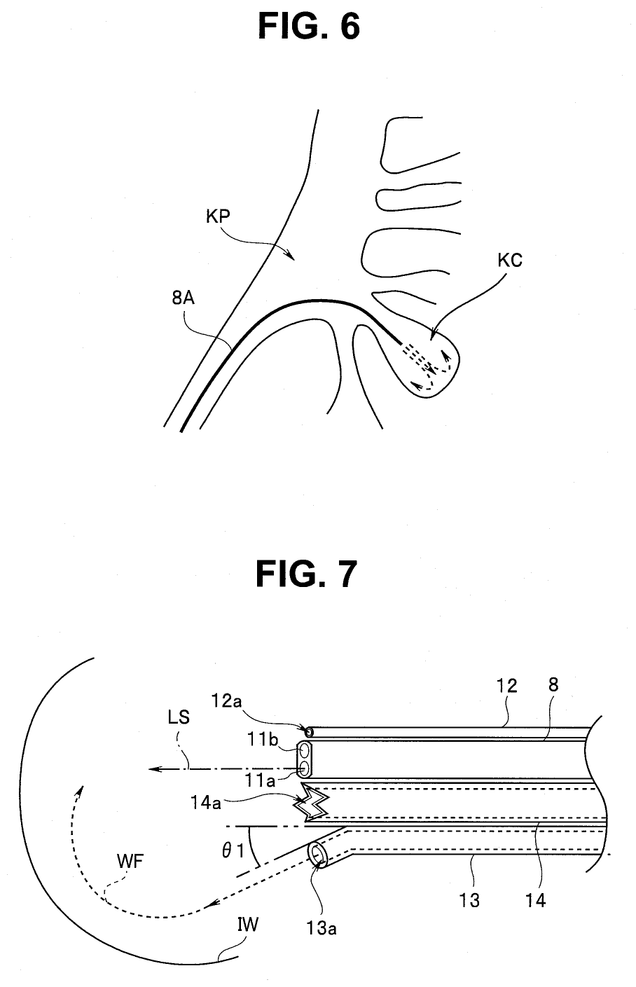

FIG. 6 is a view for explaining the flow of the physiological saline solution discharged from the distal end opening 13a of the water feeding tube 13. FIG. 6 illustrates a state where the distal end portion of the insertion portion 15 is located in a kidney calyx KC from a kidney pelvis KP of a kidney.

Since the physiological saline solution discharged from the distal end opening 13a of the water feeding tube 13 of the insertion portion 15 becomes a flow and moves in the kidney calyx KC as shown by dotted lines, the crushed calculus fragments fly up in the kidney calyx KC, and are easily sucked from the distal end opening 14a of the suction tube 14.

If the speed of the physiological saline solution discharged from the distal end opening 13a of the water feeding tube 13 is increased by increasing a rotational speed of a motor of the water feeding pump 22, the crushed calculus fragments can be caused to fly up more strongly. In other words, when a feeding speed of the physiological saline solution is enhanced, the flow reaches farther. Note that the feeding speed is a speed that does not damage a mucous membrane of the subject which is hit by the flow, or less.

Accordingly, the powdery crushed calculus fragments ride on the flow generated in the kidney, and are sucked into the distal end opening 14a of the suction tube 14 and is collected.

In other words, by the flow of the physiological saline solution discharged from the distal end opening 13a of the water feeding tube 13, the crushed calculus fragments fly up in the kidney, and therefore, the crushed calculus fragments in a wide range inside the kidney are collected.

It is difficult to collect the crushed calculus fragments over a wide range in the subject by collection only by suction.

When a size of a crushed calculus fragment that is not powdery is smaller than a size of the distal end opening 14a of the suction tube 14, the crushed calculus fragment that is not powdery is also sucked into the distal end opening 14a of the suction tube 14.

However, when the size of the crushed calculus fragment that is not powdery is smaller than the size of the distal end opening 14a of the suction tube 14, the crushed calculus fragment that is not powdery is sucked into the distal end opening 14a of the suction tube 14, but when the size of the crushed calculus fragment that is not powdery is larger than the size of the distal end opening 14a of the suction tube 14, the crushed calculus fragment that is not powdery sticks to the distal end opening 14a of the suction tube 14.

Even when the crushed calculus fragment that is not powdery sticks to the distal end opening 14a of the suction tube 14, gaps are formed between the crushed calculus fragment and the distal end opening 14a by the recessed and protruded portion 14x on the distal end surface of the suction tube 14. Therefore, the crushed calculus fragment does not completely close the distal end opening 14a, and therefore, collection of the powdery crushed calculus fragments is continued through the gaps between the crushed calculus fragment and the distal end opening 14a.

When the pressure in the conduit 32 reaches the predetermined threshold TH or less (S3: YES), the control unit 21 closes the stop valve 25 (S4). Even when the crushed calculus fragment does not completely close the distal end opening 14a, the crushed calculus fragment may close a large region of the distal end opening 14a. In such a case, the pressure in the conduit 32 reaches the predetermined threshold TH or less.

The control unit 21 closes the stop valve 25, and at a same time, sets a timer to count a predetermined time period to start count. Note that the timer may be a hardware circuit, or may be a software timer.

After the control unit 21 closes the stop valve 25, the control unit 21 determines whether or not the control unit 21 closes the stop valve 25 a predetermined times or more continuously (S5).

For example, it is determined whether or not the second stop valve 25 is continuously stopped the predetermined times, for example, five times or more continuously after the foot switch 38 is turned on.

When the second stop valve 25 is not closed the predetermined times or more continuously (S5: NO), the control unit 21 determines whether or not the timer that is set in S4 times up, and the predetermined time period elapses (S6).

When the predetermined time period does not elapse (S6: NO), no processing is performed.

When the predetermined time period lapses (S6: YES), the control unit 21 opens the stop valve 25 (S7). The control unit 21 returns to processing in S3 after S7.

When the second stop valve 25 is closed, the distal end opening 14a of the suction tube 14 does not suck the physiological saline solution, and therefore the crushed calculus fragments sticking to the distal end opening 14a are likely to separate from the distal end opening 14a due to gravity or the like.

When the crushed calculus fragments sticking to the distal end opening 14a separate from the distal end opening 14a, a detection value of the pressure gauge 36 returns to a pressure at a time of no crushed calculus fragment sticking to the distal end opening 14a.

Therefore, when the pressure exceeds the predetermined threshold in S3 (S3: NO), collection of the crushed calculus fragments is restarted.

When the second stop valve 25 closes the predetermined times or more continuously (S5: Yes), the control unit 21 executes alarm processing (S8).

When the second stop valve 25 continuously closes the predetermined times or more, the crushed calculus fragments sticking to the distal end opening 14a do not separate from the distal end opening 14a, and therefore, alarm processing that makes a notice to notify the surgeon to that effect is executed.

By the alarm processing, the surgeon separates the crushed calculus fragments sticking to the distal end opening 14a from the distal end opening 14a by different means. For example, it is also possible to rotate the motor of the suction pump 23 reversely for only a predetermined time period, after opening the stop valve 25 to cause the crushed calculus fragments to separate from the distal end opening 14a.

When the crushed calculus fragment does not completely close the distal end opening 14a, the powdery crushed calculus fragments crushed by the laser light continues to be collected.

As above, according to the aforementioned embodiment, feeding and suction are simultaneously performed, so that it is possible to collect powdery crushed calculus fragments, and it is possible to cause the crushed calculus fragments that are not powdery to stick to the distal end opening 14a of the suction tube 14 and collect the crushed calculus fragments that are not powdery.

Consequently, according to the aforementioned embodiment, it is possible to provide the endoscope and the endoscope system that can collect powdery crushed calculus fragments and the like, and can collect crushed calculus fragments and the like that are not powdery while achieving compactification of the distal end portion of the insertion portion.

Next, modifications of the aforementioned embodiment will be described.

### (Modification 1)

In the aforementioned embodiment, the physiological saline solution from the distal end opening 13a of the water feeding tube 13 is discharged in the field of view direction of the endoscope, but may be discharged in an oblique direction.

FIG. 7 is a schematic configuration diagram illustrating a configuration of a distal end portion of an insertion portion 15 relating to a present modification. As illustrated in FIG. 7, a distal end portion of a water feeding tube 13 is oriented in an oblique direction by a predetermined angle θ1 with respect to a field of view direction LS of an endoscope 5.

In other words, a discharge direction of a physiological saline solution that is delivered from the distal end portion of the water feeding tube 13 inclines by the predetermined angle θ1 with respect to the field of view direction LS parallel with a center axis of the insertion portion 15.

Accordingly, as shown by a dotted line WF, feeding is performed obliquely to a site of a subject in the field of view direction LS of a distal end portion 8a. As a result, a vortex flow is generated along an inner wall IW of the subject, and a reach range of a flow of the physiological saline solution can be made farther.

### (Modification 2)

In the aforementioned embodiment, the distal end opening 13a of the water feeding tube 13, and the distal end opening 14a of the suction tube 14 are disposed at the same position in the longitudinal axis direction of the insertion portion 15, but the distal end opening 13a of the water feeding tube 13 may be disposed on a distal end side in the longitudinal axis direction of the insertion portion 15, from the distal end opening 14a of the suction tube 14.

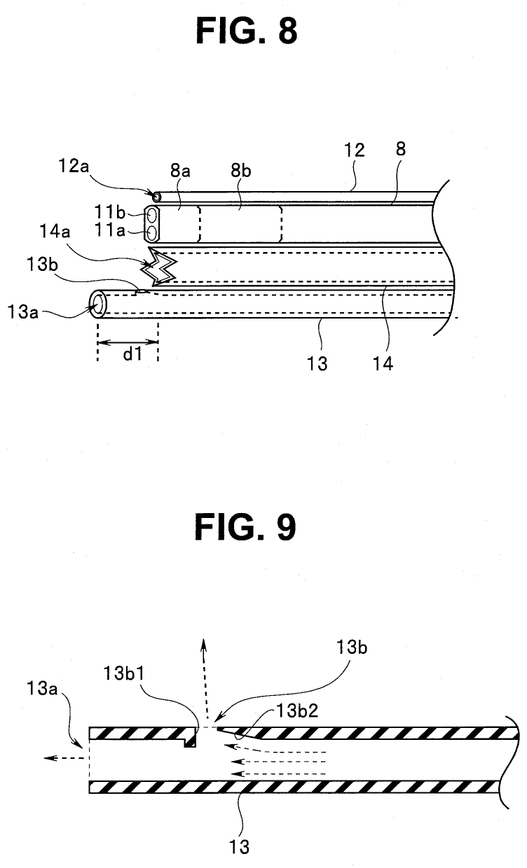

FIG. 8 is a schematic configuration diagram illustrating a configuration of an insertion portion relating to a present modification. FIG. 9 is a sectional view of a distal end portion of a water feeding tube 13 along a longitudinal axis direction of the water feeding tube 13.

As illustrated in FIG. 8, in the present modification, a distal end opening 13a of the water feeding tube 13 is disposed so as to be separated from a distal end opening 14a of a suction tube 14 in a longitudinal axis direction of an insertion portion 15 by a distance d1 toward a distal end side. In other words, the distal end opening 14a of the suction tube 14 is disposed on a proximal end side from the distal end opening 13a of the water feeding tube 13.

A lateral water feeding port 13b is formed in a side surface of a distal end portion of the water feeding tube 13. The lateral water feeding port 13b is an opening that is formed in a side surface of the water feeding tube 13 so as to be located on a distal end side from the distal end opening 14a of the suction tube 14. In other words, the lateral water feeding port 13b is a lateral opening portion that opens to a distal end opening 14a side on a distal end side from the distal end opening 14a and on a proximal end side from the distal end opening 13a.

The lateral water feeding port 13b has a lateral water feeding port wall 13b1 which is hit by a physiological saline solution flowing in the water feeding tube 13. The lateral water feeding port 13b has an inclined surface 13b2 in which a thickness of a thin walled portion becomes thinner toward a distal end side, in an inner wall surface on a proximal end side of the lateral water feeding port 13b.

In other words, the inclined surface 13b2 of the water feeding tube 13 configures a taper portion in which a thickness of a thin walled portion of the water feeding tube 13 becomes smaller toward a distal end direction of the water feeding tube 13, on a proximal end side of the lateral water feeding port 13b.

According to the configuration like this, the physiological saline solution passing through the water feeding tube 13 spreads to the lateral water feeding port 13b by the inclined surface 13b2. The physiological saline solution hits the lateral water feeding port wall 13b1, and is discharged from the lateral water feeding port 13b.

The physiological saline water is discharged from the distal end opening 13a of the water feeding tube 13, and is also discharged from the lateral water feeding port 13b in a direction orthogonal to a center axis of the suction tube 14 on a distal end side of the distal end opening 14a of the suction tube 14.

As a result, it is possible to remove crushed calculus fragments sticking to the distal end opening 14a of the suction tube 14 from the distal end opening 14a by the physiological saline solution discharged from the lateral water feeding port 13b.

### (Modification 3)

In the aforementioned embodiment, the distal end opening 13a of the water feeding tube 13 discharges the physiological saline solution in the distal end direction of the longitudinal axis of the insertion portion 15, but the discharge direction of the physiological saline solution may be changed.

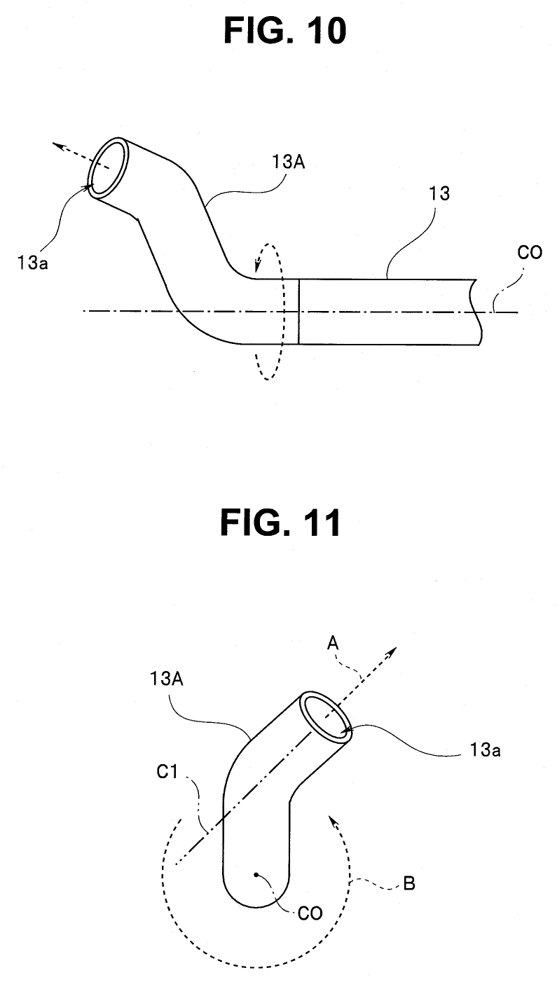

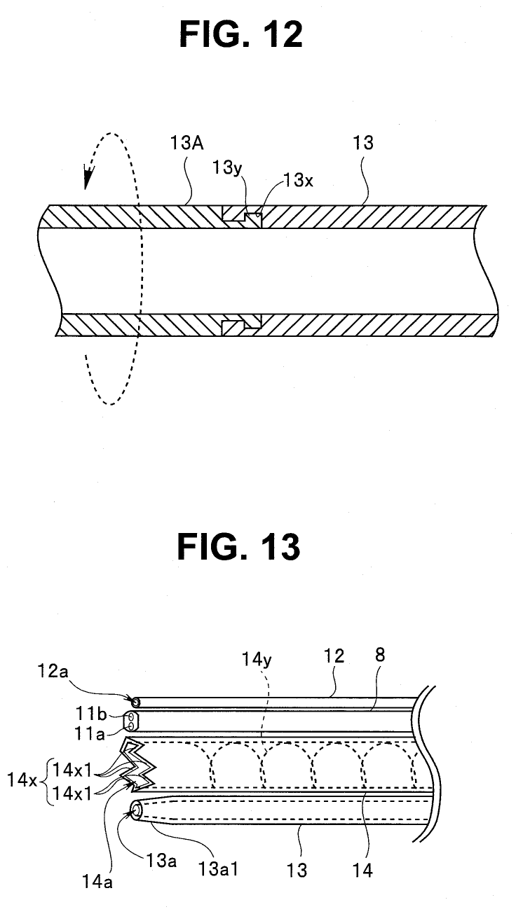

FIG. 10 is a side view of a distal end portion of a water feeding tube 13 seen in a direction orthogonal to a longitudinal axis of the water feeding tube 13 relating to a present modification. FIG. 11 is a front view of the distal end portion of the water feeding tube 13 seen in a longitudinal axis direction of the water feeding tube 13 relating to the present modification. FIG. 12 is a sectional view of connection portions of a distal end rotation portion and the water feeding tube 13 in the longitudinal axis direction of the water feeding tube 13 relating to the present modification.

As illustrated in FIG. 10, a distal end rotation portion 13A rotatable around a longitudinal axis CO of the water feeding tube 13 is provided at the distal end portion of the water feeding tube 13.

The distal end rotation portion 13A has a winding pipe shape. As illustrated in FIG. 12, an inner peripheral groove 13x is formed on an inner peripheral surface of the distal end portion of the water feeding tube 13. A circumferential protruded portion 13y that engages with the inner peripheral groove 13x is formed in a proximal end portion of the distal end rotation portion 13A.

The circumferential protruded portion 13y engages with the inner peripheral groove 13x so that the distal end rotation portion 13A is rotatable around the longitudinal axis CO of the water feeding tube 13.

A distal end opening 13a of the water feeding tube 13 is a distal end opening of the distal end rotation portion 13A.

As illustrated in FIG. 10 and FIG. 11, the distal end opening 13a of the distal end rotation portion 13A is located at a position deviated from the longitudinal axis CO of the water feeding tube 13. In the distal end opening 13a, a direction in which a physiological saline solution is discharged is formed in a direction that is obliquely forward with respect to a distal end direction of the longitudinal axis CO.

As illustrated in FIG. 11, the distal end rotation portion 13A is formed so that when the distal end rotation portion 13A is seen in the longitudinal axis direction of the water feeding tube 13 relating to the present modification, an axis C1 in a direction A in which the physiological saline solution is discharged from the distal end opening 13a does not pass through the longitudinal axis CO.

Accordingly, the distal end rotation portion 13A rotates in a direction shown by an arrow B of a dotted line by a reaction force of the physiological saline solution discharged from the distal end opening 13a, and therefore, a discharge direction of the physiological saline solution changes.

According to the configuration like this, it is possible to cause a flow of the physiological saline water to reach a wide range in the subject by solution feeding in the subject.

### (Modification 4)

In the aforementioned embodiment, the inside diameter of the water feeding tube 13 is constant from the proximal end portion to the distal end portion, but an inside diameter of the distal end portion of the water feeding tube 13 may be made smaller than an inside diameter of a proximal end portion. In other words, in the water feeding tube 13, the inside diameter of the distal end portion is formed to be smaller than the inside diameter of the proximal end portion.

FIG. 13 is a schematic configuration diagram illustrating a configuration of an insertion portion in which an inside diameter of a distal end portion of a water feeding tube 13 is made smaller than an inside diameter of a proximal end portion.

As illustrated in FIG. 13, by making the inside diameter of the distal end portion of the water feeding tube 13 smaller than the inside diameter of the proximal end portion, it is possible to generate a flow at a higher feeding speed while restricting a feeding rate of a physiological saline solution.

In other words, since it is possible to save an amount of the physiological saline solution that is fed and sucked, and obtain a fast flow even with a water feeding pump that does not have a high capacity, it is possible to achieve reduction in cost of an endoscope system 1.

When an inside of a subject is a closed space, it is also possible to prevent increase in pressure inside the subject.

### (Modification 5)

In the aforementioned embodiment, the inside diameter of the water feeding tube 13 is constant from the proximal end portion to the distal end portion, but an inside diameter of the proximal end portion of the water feeding tube 13 may be made larger than an inside diameter of the distal end portion.

By increasing the inside diameter of the proximal end portion of the water feeding tube 13, it is possible to reduce pressure loss in a water feeding path, it is possible to reduce a pressure and a capacity of a water feeding pump 22, and by extension, it is possible to reduce cost of the water feeding pump 22.

### (Modification 6)

The distal end opening 14a may be disposed in the distal end portion of the insertion portion 15 so that the distal end opening 14a of the suction tube 14 is inside an observation field of view range of the endoscope image that is obtained in the observation window 11a.

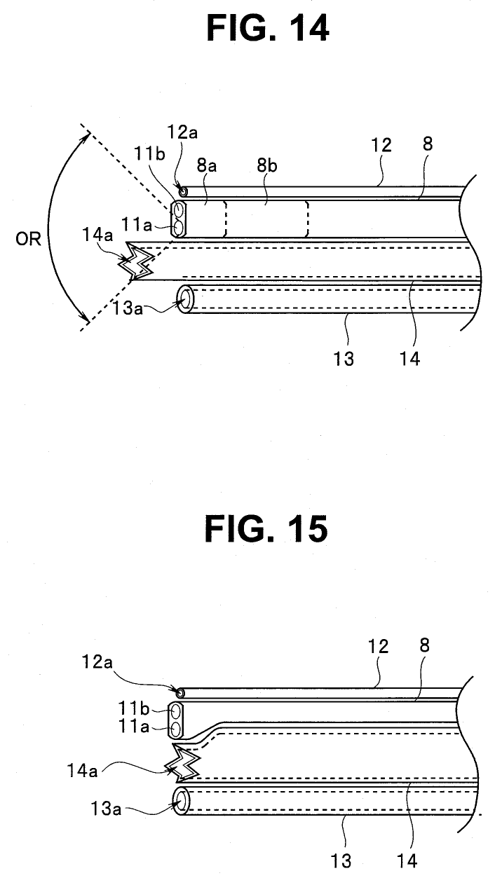

FIG. 14 is a schematic configuration diagram illustrating a configuration of an insertion portion relating to a present modification.

As illustrated in FIG. 14, a suction tube 14 protrudes in a distal end direction of a longitudinal axis of an insertion portion 15 from a distal end surface of the insertion portion 15 so that a distal end opening 4a is inside an observation field of view range by an observation window 11a. In FIG. 14, the distal end opening 14a of the suction tube 14 is inside an observation field of view range OR.

In other words, the distal end opening 14a of the suction tube 14 is disposed to protrude to a distal end side from the distal end surface formed in the insertion portion 15 so as to be disposed inside the observation field of view range of the observation window 11a.

Therefore, according to the present modification, a surgeon can confirm whether a crushed calculus fragment, a mucous membrane in a subject or the like is sticking to the distal end opening 14a of the suction tube 14 by looking at an endoscope image. As a result, the surgeon can remove the sticking crushed calculus fragment, mucous membrane in the subject or the like from the distal end opening 14a by closing the stop valve 25, stopping the suction pump 23 or the like.

Thereby, it is possible to prevent a damage to a mucous membrane by suction. When an inside of the subject is a closed space, it is also possible to prevent increase in pressure inside the subject due to reduction in suction amount.

### (Modification 7)

In the aforementioned embodiment, the suction tube 14 has the same inside diameter from the distal end to the proximal end, but an inside diameter of the distal end portion of the suction tube 14 may be made smaller than an inside diameter of the proximal end portion.

FIG. 15 is a schematic configuration diagram illustrating a configuration of an insertion portion relating to a present modification.

As illustrated in FIG. 15, an inside diameter of a part including a distal end opening 14a of a suction tube 14 is smaller than an inside diameter of a proximal end portion of the suction tube 14. In other words, in the suction tube 14, an inside diameter on a distal end side is formed to be smaller than an inside diameter on a proximal end side.

According to the configuration like this, it is possible to prevent clogging of the suction tube 14 by preventing entry of a large crushed calculus fragment into the suction tube 14 from the distal end opening 14a.

In particular, when an endoscope is a flexible endoscope and the endoscope bends, a sectional shape deforms on the proximal end side of the suction tube 14, a minimum inside diameter decreases, and clogging with crushed calculus fragments easily occurs, so that increasing the inside diameter on the proximal end side in advance is effective in prevention of clogging of the suction tube 14.

### (Modification 8)

In the aforementioned embodiment, the shape of the distal end opening 14a of the suction tube 14 is circular, but may be an elliptical shape.

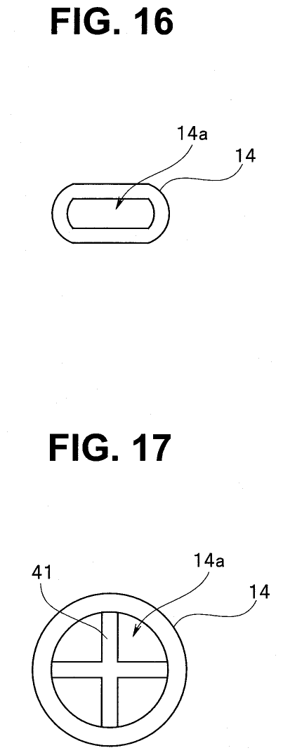

FIG. 16 is a front view illustrating a shape of a distal end opening 14a of a suction tube 14 relating to a present modification.

As illustrated in FIG. 16, the distal end opening 14a of the suction tube 14 of the present modification does not have the recessed and protruded portion 14x as described above, but has an elliptical shape.

By being formed into the shape like this, the distal end opening 14a is narrowed, and it is possible to prevent clogging in the suction tube 14 with crushed calculus fragments.

Note that the distal end opening 14a of the suction tube 14 may have an elliptical shape and also have the recessed and protruded portion 14x as described above.

### (Modification 9)

In the aforementioned embodiment, in the distal end opening 14a of the suction tube 14, nothing is provided, but a partition member or the like may be provided.

FIG. 17 is a front view of a distal end opening 14a of a suction tube 14 relating to a present modification.

As illustrated in FIG. 17, the distal end opening 14a of the suction tube 14 of the present modification does not have the recessed and protruded portion 14x as described above, but has a partition member 41. The partition member 41 is fixed to the distal end opening 14a of the suction tube 14 by an adhesive or the like.

Here, the partition member 41 is made of a metal or a resin, and is in a cross shape, but may be a member in a single bar shape or a plate shape in a vertical direction without having a part in a lateral direction, for example.

By adopting the shape like this, the distal end opening 14a is narrowed, and therefore it is possible to prevent clogging in the suction tube 14 with crushed calculus fragments.

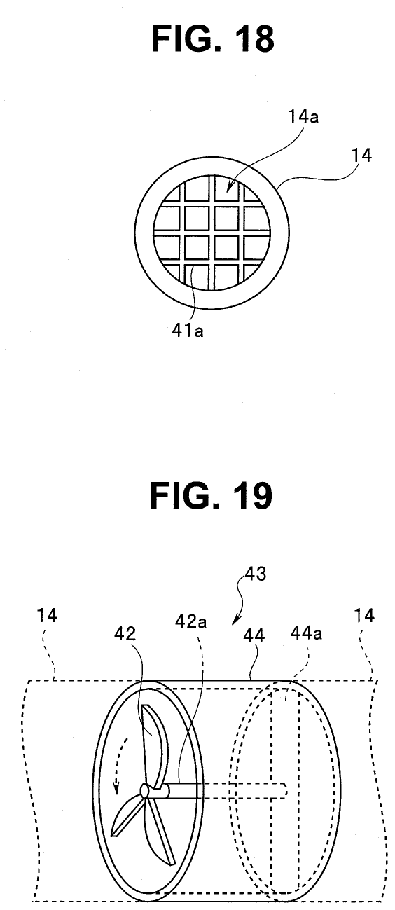

Note that here, the partition member 41 is a cross-shaped member, but may be a net-shaped member 41a as illustrated in FIG. 18. FIG. 18 is a front view of the distal end opening 14a of the suction tube 14 relating to the present modification.

### (Modification 10)

A vibration generator may be provided in each of a water feeding path and a suction path.

In FIG. 1, as shown by dotted lines, ultrasound transducers 37A and 37B as vibration generators are respectively provided to be closely attached to a conduit 31 and a conduit 32.

The ultrasound transducer 37A provided at the conduit 31 is connected to a control unit 21 by a signal line, and gives ultrasound vibration to the conduit 31 in response to a control signal from the control unit 21. The ultrasound vibration given to the conduit 31 is also transmitted to a water feeding tube 13.

Likewise, the ultrasound transducer 37B provided at the conduit 32 is connected to the control unit 21 by a signal line, and gives ultrasound vibration to the conduit 32 in response to a control signal from the control unit 21. The ultrasound vibration given to the conduit 32 is also transmitted to a suction tube 14.

By doing so, it is possible to reduce pressure losses in the water feeding path formed by the water feeding tube 13 and the conduit 31, and in the suction path formed by the suction tube 14 and the conduit 32.

As a result, it is possible to reduce pressure and capacity of a water feeding pump 22, and by extension, it is possible to reduce cost of the water feeding pump 22.

In particular, the ultrasound transducer 37B can also contribute to prevention of clogging of the suction tube 14.

Note that here, ultrasound vibration is used, but vibration does not have to be ultrasound vibration.

Note that the vibration generator may be provided at either one of the water feeding path and the suction path.

As above, the ultrasound transducer as the vibration generator that gives at least one of the water feeding tube 13 and the suction tube 14 may be provided.

### (Modification 11)

In the aforementioned embodiment, in the suction tube 14, nothing is provided, but a propeller that generates a vortex may be provided.

FIG. 19 is a view illustrating a propeller provided in a suction tube 14, relating to a present modification.

As illustrated in FIG. 19, a propeller unit 43 having a propeller 42 made by a MEMS (micro electro mechanical systems) technology is provided in the suction tube 14 or midway in the suction tube 14.

A shaft 42a of the propeller 42 is fixed to a support plate 44a fixed to an inside of an annular support member 44. The propeller 42 is attached to a distal end portion of the shaft 42a rotatably with respect to the shaft 42a. The support member 44 is fixed to the suction tube 14 by an adhesive or the like, midway in the suction tube 14.

Accordingly, a physiological saline solution flowing in the suction tube 14 rotates the propeller 42 as shown by an arrow of a dotted line, and as a result, a flow of the physiological saline solution becomes a vortex flow. Accordingly, the physiological saline solution becomes a vortex flow in the suction tube 14 and is sucked.

### (Modification 12)

In the aforementioned embodiment, feeding and suction of the physiological saline solution is continuously performed when the foot switch 38 is turned on, but suction may be performed intermittently.

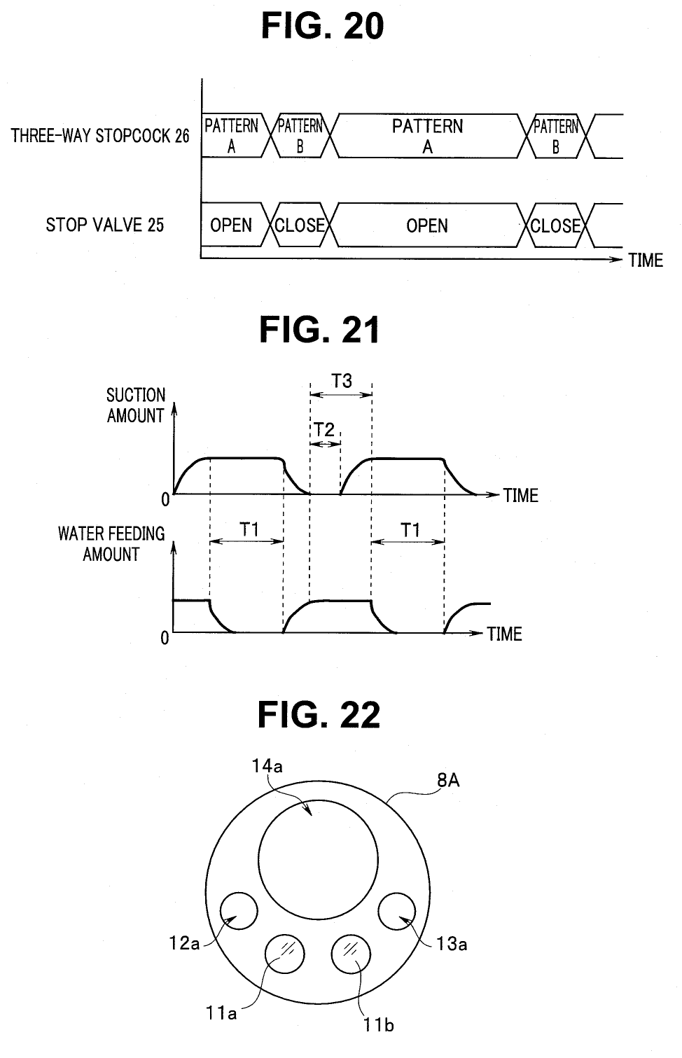

FIG. 20 is a time chart of operations of a stop valve and a three-way stopcock for intermittent suction of suction relating to a present modification.

A horizontal axis in FIG. 20 represents a time. A state of a three-way stopcock 26 switches between patterns A and B at a predetermined timing. As illustrated in FIG. 20, a control unit 21 controls the three-way stopcock 26 so that a time period of the pattern A becomes longer than a time period of the pattern B.

When the three-way stopcock 26 is in the pattern A, a physiological saline solution from a conduit 31 is supplied to a water feeding tube 13. When the three-way stopcock 26 is in the pattern B, the physiological saline solution from the conduit 31 is supplied to a conduit 33.

As illustrated in FIG. 20, the stop valve 25 is controlled by the control unit 21 so as to open when the three-way stopcock 26 is in the pattern A, and to be closed when the three-way stopcock 26 is in the pattern B.

At a time of the pattern A, a physiological saline solution from a water feeding pump 22 passes through the three-way stopcock 26 and is supplied to the water feeding tube 13, and a physiological saline water from a suction tube 14 passes through the stop valve 25 and is supplied to a suction tank 28.

At a time of the pattern B, the physiological saline solution from the water feeding pump 22 flows to the conduit 33 from the three-way stopcock 26, but since the stop valve 25 is closed, the physiological saline solution flows from the conduit 33 to the suction tube 14. As a result, the physiological saline solution is discharged from a distal end opening 14a of the suction tube 14, when the three-way stopcock 26 is in the pattern B.

In other words, the control unit 21 controls the state of the three-way stopcock 26 so that delivery of the physiological saline solution is performed from the suction tube 14 when delivery of the physiological saline solution from the water feeding tube 13 is stopped.

Accordingly, crushed calculus fragments sticking to the distal end opening 14a of the suction tube 14 are removed by the physiological saline solution that is intermittently discharged from the distal end opening 14a.

Note that since the physiological saline solution is not sucked and pressure inside a subject increases at the time of the pattern B, a suction amount immediately after the pattern is switched to the pattern A may be temporarily increased, or a water feeding amount immediately after the pattern is switched to the pattern A may be temporarily decreased. As a result, it is possible to restrict an increase in the physiological saline water amount in the subject and a pressure increase in the subject in a short time period.

Since the physiological saline solution is not sucked and the pressure in the subject increases at the time of the pattern B, a suction amount immediately before the pattern is switched to the pattern B may be temporarily increased, or a water feeding amount immediately before the pattern is switched to the pattern B may be temporarily decreased. As a result, it is possible to restrict the increase in the physiological saline solution amount in the subject and the pressure increase in the subject in a short time period.

At the time of the aforementioned pattern B, the stop valve 25 may be kept open, or the stop valve 25 may be caused not to be closed completely. In doing so, a feeding speed of the physiological saline solution from the suction tube 14 is restricted, and it is possible to prevent crushed calculus fragments from popping out of the suction tube 14 at a high speed.

When the stop valve 25 is closed, all of the three paths of the three-way stopcock 26 may be opened. In doing so, the feeding speed of the physiological saline solution from the suction tube 14 is restricted, and it is possible to prevent the crushed calculus fragments from popping out of the suction tube 14 at a high speed. In doing so, especially in modification 2 described above, it is also possible to cause feeding from the lateral water feeding port 13b.

### (Modification 13)

In the aforementioned embodiment, feeding and suction of the physiological saline solution are continuously performed when the foot switch 38 is turned on, but the feeding and suction may be alternately performed.

FIG. 21 is a time chart of operations of feeding and suction relating to a present modification.

A horizontal axis in FIG. 21 represents a time. Timings for feeding and suction are controlled so that when feeding by the water feeding pump 22 is not performed, suction by the suction pump 23 is performed, and when suction by the suction pump 23 is not performed, feeding by the water feeding pump 22 is performed.

In FIG. 21, the control unit 21 controls the water feeding pump 22 and the suction pump 23 so that the suction pump 23 drives before a timing when the water feeding pump 22 stops, and the water feeding pump 22 drives at a same time as the suction pump 23 stops.

In other words, the control unit 21 controls a feeding operation by the water feeding tube 13 and a suction operation by the suction tube 14, so that a feeding operation by the water feeding tube 13 is performed during stoppage of the suction operation by the suction tube 14.

In a period T1 illustrated in FIG. 21, a discharge amount of the physiological saline solution from the distal end opening 13a of the water feeding tube 13 changes, so that a size and flow of the crushed calculus fragments flying up in the subject by feeding change, and various crushed calculus fragments can be sucked from the distal end opening 14a of the suction tube 14.

Since suction is not performed in a period T2, the crushed calculus fragments sticking to the distal end opening 14a of the suction tube 14 drop in a gravity direction, or can be separated from the distal end opening 14a by a flow of water generated in the subject due to the physiological saline solution discharged from the distal end opening 13a of the water feeding tube 13.

In a period T3, suction is not performed, or the suction amount is small, and therefore the crushed calculus fragments can be caused to fly up in the subject by feeding.

Note that in modification 2 described with FIG. 8 and FIG. 9 described above, feeding and suction may be alternately performed. In that case, the water feeding amount is small or feeding is not performed in the period T1, and therefore, the crushed calculus fragments can be effectively sucked from the distal end opening 14a of the suction tube 14 without being hindered by the lateral feeding from the lateral water feeding port 13b. Since feeding from the lateral water feeding port 13b is performed in the period T2 in which suction is not performed, the crushed calculus fragments in the distal end opening 14a of the suction tube 14 are easy to fly.

### (Modification 14)

In the aforementioned embodiment, the insertion portion 15 includes the endoscope insertion portion 8, but the channel tube 12, the water feeding tube 13 and the suction tube 14 may be placed in the endoscope insertion portion 8 by increasing an outside diameter of the endoscope insertion portion 8.

FIG. 22 is a configuration diagram of a distal end portion of an endoscope insertion portion 8A as the distal end portion of the endoscope insertion portion 8A is seen in a distal end direction of a longitudinal axis of the endoscope insertion portion 8A as an insertion portion 15, relating to a present modification.

As illustrated in FIG. 22, an observation window 11a, an illumination window 11b, a distal end opening 12a of a treatment instrument insertion channel, a distal end opening 13a of a water feeding channel and a distal end opening 14a of a suction channel are provided in a distal end surface of the endoscope insertion portion 8A.

By the configuration like this, a similar effect to the effect of the aforementioned embodiment is provided.

### (Modification 15)

In the aforementioned embodiment, the example of feeding and suction of a liquid (physiological saline solution in this case) is cited as a fluid, but feeding and suction of a liquid other than the physiological saline solution, or gas such as carbon dioxide may be performed.

Accordingly, the endoscope system of the aforementioned embodiment is also applicable to collection of living tissue that is excised in a treatment to a nasal cavity, a womb, a bladder or the like.

### (Modification 16)

In the aforementioned embodiment, collection of crushed calculus fragments by the laser light is described, but the endoscope system of the aforementioned embodiment is also applicable to collection of very small calculus fragments to which calculus crushing or the like is not performed.

In other words, the endoscope system of the aforementioned embodiment is also applicable to collection of living tissue of a polyp, a myoma and the like other than a calculus.

### (Modification 17)

In the aforementioned embodiment, the single insertion portion 15 is formed by closely attaching and fixing the endoscope insertion portion 8, the channel tube 12, the water feeding tube 13 and the suction tube 14 to one another in the bundled state by the fixing means such as an adhesive, within the range LL corresponding to the length inserted into the subject, but only the distal end portions may be fixed.

By doing so, an endoscope insertion portion 8, a channel tube 12, a water feeding tube 13 and a suction tube 14 move independently in proximal end portions that are not fixed, so that when the endoscope is bent, or when a flexible portion bends along a shape of a subject, the respective tubes can change a positional relationship, a force that is applied to the tubes is reduced, and deformation of tube sectional shapes and buckling hardly occur.

### (Modification 18)

In the aforementioned embodiment, the single insertion portion 15 is formed by bringing a total of four that are the endoscope insertion portion 8, the channel tube 12, the water feeding tube 13, and the suction tube 14 into a bundled state, but the water feeding tube 13 or the suction tube 14 may be used as the channel tube.

In other words, a single insertion portion 15 is formed by bringing a total of three that are an endoscope insertion portion 8, a water feeding tube 13, and a suction tube 14 into a bundled state, and a treatment instrument such as a laser probe 3a may be inserted into the water feeding tube 13 or the suction tube 14 and used.

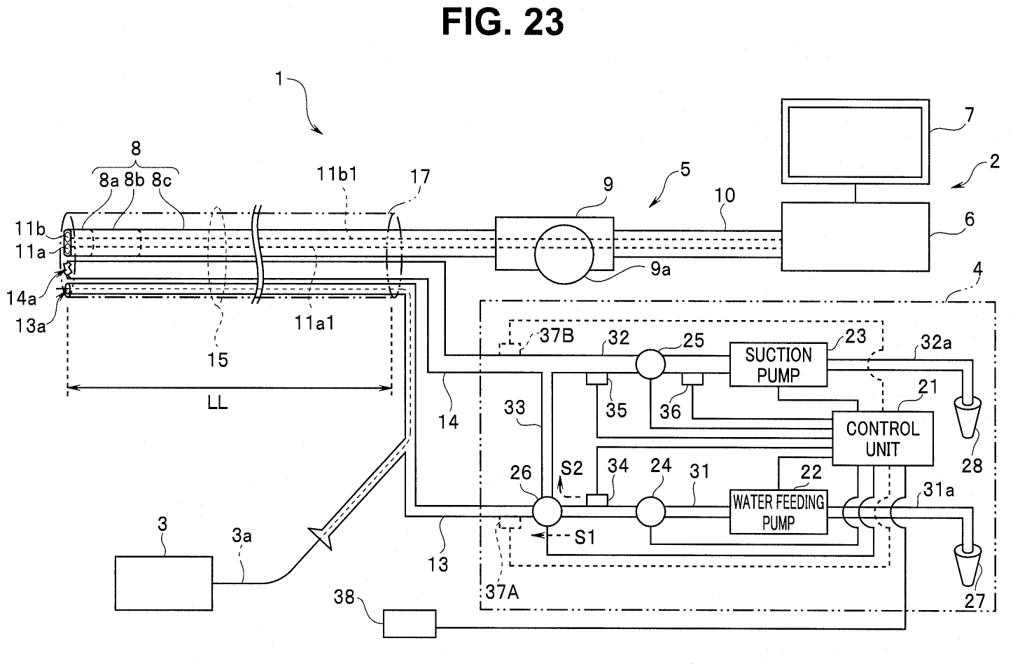

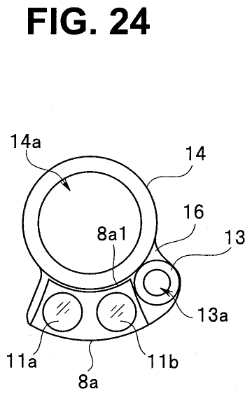

FIG. 23 is a configuration diagram of an endoscope system relating to present modification 18. In FIG. 23, same components as in FIG. 1 are assigned with the same reference signs, and explanation will be omitted. FIG. 24 is a configuration diagram of a distal end portion of the insertion portion 15 as the distal end portion of the insertion portion 15 is seen in a distal end direction of a longitudinal axis of the insertion portion 15.

In FIG. 23, the water feeding tube 13 has a branch tube for inserting a treatment instrument from midway. A laser probe 3a as the treatment instrument is inserted through an inside of the water feeding tube 13 from an end portion opening of the branch tube.

By doing so, as illustrated in FIG. 24, it is possible to realize a finer insertion portion diameter. Further, it is also possible to eliminate clogging of the suction tube 14 with the treatment instrument, when an inside of the suction tube 14 is clogged with crushed calculus fragments.

### (Modification 19)

In the aforementioned embodiment, the control unit 21 determines whether or not the pressure in the conduit 32 has the predetermined threshold TH or less based on the detection signal of the pressure gauge 36, but the control unit may determine whether or not the flow rate in the conduit 32 has the threshold TH or less based on a detection signal from the flowmeter 35.

In other words, when a wide region of a distal end opening 14a is clogged with crushed calculus fragments, a flow rate in a conduit 32 has a predetermined threshold TH or less even when a suction pump 23 increases a suction force to a specified upper limit to keep the flow rate in the conduit 32 constant. When the flow rate in the conduit 32 has the predetermined threshold TH or less, a control unit 21 closes a stop valve 25 (S4).

By doing so, it is possible to separate the crushed calculus fragments sticking to the distal end opening 14a from the distal end opening 14a without using a pressure gauge 36, and it is also possible to eliminate clogging of the suction tube 14.

By doing so, it is possible to provide the pressure gauge 36 in a conduit 31 instead of the conduit 32, and it is also possible to detect a pressure in a subject correctly even when the suction tube 14 or the conduit 32 is clogged with crushed calculus fragments. Thereby, the control unit 21 can control a water feeding pump 22 or a suction pump 23 based on a detection signal of the pressure gauge 36 so that the pressure in the subject does not reach a predetermined value or more for safety of the subject, even when an inside of a suction path is clogged with crushed calculus fragments.

### (Modification 20)

In the aforementioned embodiment, the control unit 21 determines whether or not the pressure in the conduit 32 has the predetermined threshold TH or less based on the detection signal of the pressure gauge 36 in S3 in FIG. 5, but the control unit 21 may determine whether or not a suction force of the suction pump 23 reaches the predetermined threshold TH or more based on a control signal of the suction pump 35.

In other words, when a wide region of a distal end opening 14a is clogged with crushed calculus fragments, a suction pump 23 increases a suction force to a predetermined threshold TH or more to keep a flow rate in a suction tube 32 constant. When the suction force of the suction pump 23 reaches the predetermined threshold TH or more, a control unit 21 closes a stop valve 25 (S4).

By doing so, a similar effect to the effect of modification 19 is obtained.

As above, according to the aforementioned embodiment and respective modifications, it is possible to provide an endoscope and an endoscope system capable of performing collection of powdery crushed calculus fragments or the like, and collecting crushed calculus fragments that are not powdery while achieving compactification of the distal end portion of the insertion portion.

The present disclosure is not limited to the aforementioned embodiment, and various modifications, alterations and the like can be made within the range without departing from the gist of the present disclosure.

1. An endoscope comprising:
an insertion portion configured to be inserted into a subject;
a first conduit that includes a first distal end opening provided in a distal end portion of the insertion portion, is disposed along a longitudinal axis of the insertion portion from the distal end portion of the insertion portion, and is capable of delivering a fluid; and
a second conduit that includes a second distal end opening provided in the distal end portion of the insertion portion and different from the first distal end opening, is disposed along the longitudinal axis of the insertion portion from the distal end portion of the insertion portion, and is capable of suctioning the fluid,
wherein:
the second distal end opening has an opening area larger than an opening area of the first distal end opening, and
in the second conduit, an inside diameter on a distal end side is smaller than an inside diameter on a proximal end side.

2. The endoscope according to claim 1, further comprising a third conduit that includes a third distal end opening provided in the distal end portion of the insertion portion and different from the first distal end opening and the second distal end opening, and is disposed along the longitudinal axis of the insertion portion from the distal end portion of the insertion portion.

3. The endoscope according to claim 1, wherein a distal end surface of the second conduit distal end opening includes a protruded portion or a recessed portion that is respectively protruded or recessed in a direction of the longitudinal axis.

4. The endoscope according to claim 1, wherein:
the distal end portion of the insertion portion includes an observation window for observing the subject, and
a distal end of the second conduit is disposed to protrude in a distal direction along the longitudinal axis from a distal end surface of the insertion portion such that the second distal end opening is disposed in an observation field of view range of the observation window.

5. The endoscope according to claim 1, wherein:
the first distal end opening is disposed to protrude to a distal of end side from the second distal end opening, and
the first conduit further includes a lateral opening portion that opens toward the second distal end opening at a position proximal of the first distal opening and distal of the second distal opening.

6. The endoscope according to claim 5, wherein the first conduit includes a tapered portion in which a thickness of a portion of a wall of the first conduit decreases in a distal direction of the first conduit, at a position proximal of the lateral opening portion.

7. The endoscope according to claim 1, wherein the second conduit is configured to generate a vortex flow when suctioning the fluid inside the second conduit.

8. The endoscope according to claim 1, further comprising:
a connection portion configured to connect a proximal end portion of the first conduit and a proximal end portion of the second conduit, and
a three-way stopcock that is provided in the connection portion.

9. The endoscope according to claim 1, wherein the first conduit is configured to discharge the fluid from a distal end portion thereof in a discharge direction that is inclined by a predetermined angle with respect to a center axis of the insertion portion.

10. The endoscope according to claim 1, wherein in the first conduit, an inside diameter of a distal end portion smaller than an inside diameter of a proximal end portion.

11. The endoscope according to claim 1, further comprising a vibration generator configured to transmit vibration to at least one of the first conduit and the second conduit.

12. An endoscope system, comprising:
the endoscope according to claim 1; and
a processor configured to control a first pump that is configured to deliver the fluid to the first conduit, and a second pump that is configured to suction the fluid from the second conduit.

13. The endoscope system according to claim 12, wherein the processor is further configured to control delivery of the fluid by the first conduit, and suction of the fluid by the second conduit so that the delivery of the fluid by the first conduit is performed while the suction of the fluid by the second conduit is stopped.

14. The endoscope system according to claim 12, further comprising:
a connection portion configured to connect a proximal end portion of the first conduit and a proximal end portion of the second conduit; and
a three-way stopcock provided in the connection portion,
wherein the processor is configured to control a state of the three-way stopcock so as to deliver the fluid from the second conduit when delivery of the fluid from the first conduit is stopped.

15. The endoscope system according to claim 12, wherein the processor is configured to control the first pump or the second pump based on a pressure in the second conduit so that the pressure does not increase to a predetermined value or more.

16. The endoscope system according to claim 12, wherein the processor is configured to control the first pump or the second pump so as to:
start suctioning the fluid by the second conduit before the first conduit stops delivering the fluid, and
start delivering the fluid by the first conduit at a substantially same time the second conduit stops suctioning the fluid.

17. An insertion portion of an endoscope, the insertion portion being configured to be inserted into a subject and comprising:
a first conduit that includes a first distal end opening provided in a distal end portion of the insertion portion, is disposed along a longitudinal axis of the insertion portion from the distal end portion of the insertion portion, and is capable of delivering a fluid; and
a second conduit that includes a second distal end opening provided in the distal end portion of the insertion portion and different from the first distal end opening, is disposed along the longitudinal axis of the insertion portion from the distal end portion of the insertion portion, and is capable of suctioning the fluid,
wherein:
the second distal end opening has an opening area larger than an opening area of the first distal end opening, and
in the second conduit, an inside diameter on a distal end side is smaller than an inside diameter on a proximal end side.

18. A method for collecting a calculus in a subject comprising:
delivering a fluid into the subject from a first conduit, and suctioning the fluid through a second conduit as a first operation, and
stopping delivery of the fluid from the first conduit, and delivering the fluid from the second conduit as a second operation,
wherein the first operation and the second operation are repeated so that the first operation becomes longer than the second operation.

19. The method according to claim 18, further comprising:
detecting whether or not a pressure in the subject is equal to or greater than a threshold pressure, wherein
when the pressure is equal to or greater than the threshold pressure, the second operation is performed.

20. The method according to claim 18, wherein:
the calculus is crushed by delivering the fluid from the first conduit, and crushed calculus fragments are suctioned with the fluid by the second conduit during the first operation, and
sticking calculus fragments that are stuck to a distal end opening of the second conduit are removed by performing the second operation.
